(19) United States
(12) Patent Application Publication
(10) Pub. No.: US 2026/0324568 A1
(43) Pub. Date: Oct. 22, 2026

(54) AUTOMATED PLANT WATERING SYSTEM

(71) Applicant: JACOB ERLING JANIKSELA, CROSSLAKE, MN (US)

(72) Inventor: JACOB ERLING JANIKSELA, CROSSLAKE, MN (US)

(21) Appl. No.: 19/181,576

(22) Filed: Apr. 17, 2025

(51) Int. Cl. A01G 27/00; A01G 9/04; A01G 9/24; G01N 33/24; G05D 7/06

(52) U.S. Cl. CPC A01G 27/003; A01G 9/04; A01G 9/247; A01G 27/008; G01N 33/246; G05D 7/0635

(57) ABSTRACT

An automated plant watering system comprises a control panel, a valve panel that is electrically connected to the control panel and in fluid communication with a water source, and a plurality of watering zone sensor devices which are electrically connected to the control panel through the valve panel and in fluid communication with the valve panel. A method of plant watering with the automated plant watering system utilizes signals from the watering zone sensor devices to selectively provide water to a plurality of watering zones based upon a user's input on the control panel.

### FIELD OF THE INVENTION

The present application relates to automated plant watering systems.

### DISCUSSION OF THE RELATED ART

A number of automated plant watering systems have been previously proposed. Examples of existing automated plant watering systems range from large-scale irrigation systems for watering lawns, golf courses, agricultural fields and the like, to small-scale plant watering pots for watering individual plants. The large-scale irrigation systems can be overly complex and cumbersome to install and operate for many users. These large-scale systems are also typically installed outdoors where water damage is not a concern. The small-scale plant watering pots have various drawbacks. Typically, they integrate the controls, sensors, water supply, and a plant pot with multiple chambers into one unit. The small-scale plant watering pots cannot be interchanged with any other standard plant pots, due to their integrated nature, which may be problematic if a particular decorative pot is desired or if the plant grows large enough to require a larger plant pot. Another drawback to the plant watering pots is that the user has to monitor each pot individually, oftentimes having to refill a water reservoir located within the plant pot.

### BRIEF SUMMARY OF THE INVENTION

To overcome challenges introduced in the background, an automated plant watering system is proposed. The automated plant watering system comprising a control panel, a valve panel that is electrically connected to the control panel and in fluid communication with a water source, and a plurality of watering zone sensor devices which are electrically connected to the control panel through the valve panel and in fluid communication with the valve panel. A method of plant watering with the automated plant watering system utilizes signals from the watering zone sensor devices to selectively provide water to a plurality of watering zones based upon a user's input on the control panel. The watering zone comprising a standard plant pot containing soil, and a plant saucer, also referred to as a drip tray. The standard plant pot is any readily available commercial plant pot of any size, shape, or material of construction. The plant pot is positioned in relation to the plant saucer so that the plant saucer collects excess water that drains from the plant pot. The watering zone sensor devices incorporate soil moisture sensors which are adapted to be placed in the soil of the plant pot to monitor the moisture level of the soil. The watering zone sensor devices also incorporate water level sensors, said sensors being located in the watering zone so as to monitor the water level in the plant saucer. In at least one embodiment, each watering zone may be watered on a calendar schedule instead of utilizing the moisture sensors. In at least one embodiment, the system utilizes signals from other environmental sensors in order to determine a watering schedule. For example, sensors that detect light, environmental humidity, alkalinity, salinity, fertility, and temperature, which all affect a plant's health may be incorporated into the system.

The automated plant watering system of the present application can be installed indoors with minimal risk of water damaging the interior environment, as any excess water is collected in a plant saucer, and watering is terminated should the water in the plant saucer reach a high level. Many of the previously proposed irrigation controllers contain dozens of options for controlling a watering system as part of the user interface. The present application has a refined set of options for controlling the watering system, thus streamlining configuration of the system to meet the user's needs.

The automated plant watering system of the present application is able to provide water to a plurality of plant watering zones with a single control unit, unlike the plant watering pots, which utilize a control unit for each watering zone. The watering zone devices of the present application can be installed in existing standard plant pots and are in fluid communication with a water source external to the plant pot so that the water supply does not need to be monitored.

### BRIEF DESCRIPTION OF THE DRAWINGS

FIG. 1 is a block diagram of the automated plant watering system.

FIG. 2 is a diagram of the automated plant watering system showing a plurality of watering zones.

FIG. 3 is a diagram of the watering zone sensor device according to the preferred embodiment installed in a watering zone.

FIG. 4 is a section view of the watering zone sensor device according to the preferred embodiment installed in a watering zone.

FIG. 5 is a diagram of the watering zone sensor device according to an alternative embodiment installed in a watering zone.

FIG. 6 is a section view of the watering zone sensor device according to an alternative embodiment installed in a watering zone.

FIG. 7 is a diagram showing elements of the user interface.

FIG. 8 is a flow chart of the soil moisture range selection.

FIG. 9 is a flow chart of the automated plant watering system watering process.

### DETAILED DESCRIPTION

The following description provides details and examples set forth in the background and summary in order to provide a better understanding of the various embodiments of the automated plant watering system and should not be taken in a limiting sense. The scope of the present application should be determined with reference to the claims.

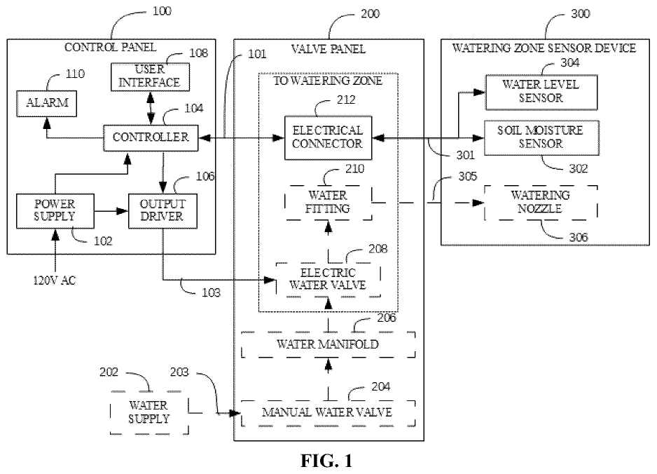

Referring to FIG. 1, a block diagram is shown illustrating an automated plant watering system according to the preferred embodiment. The system comprising a control panel 100, a valve panel 200 which is electrically connected to the control panel 100, said valve panel also in fluid communication with a water source 202, and a watering zone sensor device 300, which is electrically connected to the control panel 100 through the valve panel 200, said watering zone sensor device also in fluid communication with an electrically operated water valve 202 located in the valve panel 200. In at least one embodiment, the control panel 100 and valve panel 200 are combined into a single unit. In at least one embodiment, the control panel 100 is electrically connected to a plurality of valve panels 200. In the preferred embodiment, the output driver 106 of the control panel 100 is electrically connected to the electrically operated water valves 208 of the valve panel 200 with a first multi-conductor electrical cable 103. In the preferred embodiment, the controller 104 of the control panel 100 is electrically connected to the electrical connector 212 of the valve panel 200 with a second multi-conductor electrical cable 101. In at least one other embodiment, the control panel 100 is in wireless communication with the valve panel 200. In the preferred embodiment, the watering nozzles 306 of the watering zone sensor devices 300 are in fluid communication with the water fittings 210 of the valve panel 200 using water tubing. The watering zone sensor devices 300 utilize a third multi-conductor cable 301 to electrically connect the soil moisture sensors 302 and the water level sensors 304 to the electrical connectors 212 of the valve panel 200 in the preferred embodiment. In at least one embodiment, the watering zone sensor devices 300 are in wireless communication with the control panel 100. In at least one embodiment, the watering zone sensor devices 300 are in wireless communication with the valve panel 200.

In the preferred embodiment, the control panel 100 comprises a power supply 102, controller 104, output driver 106, user interface 108, and alarm 110. In the preferred embodiment, the control panel 100 receives power from a standard 120V AC household electrical outlet and the power supply 102 converts the 120V AC power to a voltage required by the system. In at least one embodiment, the control panel receives power that is of a different voltage, such as 240V AC. In at least one embodiment, power is provided by different means, such as a direct current battery, solar panel, or wind turbine. The power supply 102 in the preferred embodiment converts the 120V AC power to 12V DC, but a power supply 102 with a different output voltage or alternating current may be utilized, such as 24V DC. The power supply 102 provides power to the controller 104 and the output driver 106. The output driver 106 in the preferred embodiment consists of circuitry that receives low voltage signals from the controller 104 to supply higher voltage power to the electrically operated water valves 202 in the valve panel 200. In at least one embodiment, the output driver is utilized to provide power to the watering zone sensor devices 300. In the preferred embodiment, the controller 104 is a programmable microcontroller and is programmed to selectively provide water to a plurality of watering zones based on input from the user interface 108 and signals received from a plurality of watering zone sensor devices 300. The controller 104 outputs a signal to an output driver 106, the output driver 106 then provides power to the electrically operated water valves 202 in the valve panel 200. In at least one embodiment, the controller 104 has a wireless connection with an external device in order to remotely monitor and operate the system. In at least one embodiment, the controller 104 records system events and stores that information to an external data storage device, such as a hard disk drive. In the preferred embodiment, the user interface 108 is a graphical user interface displayed on a touchscreen LCD. In at least one embodiment, the user interface consists of a display and physical buttons. An alarm 110 is activated when the plant watering system encounters an error, such as if the water level sensor 304 indicates a high water level. The alarm consists of a piezoelectric sound device and LED light which are mounted to the exterior of the control panel 100 in the preferred embodiment, but may consist of various other elements to warn the user that an error has occurred. In at least one embodiment, the control panel is in wired or wireless communication with an alarm external to the system.

In the preferred embodiment, the valve panel 200 comprises a manual water valve 204, a water manifold 206, a plurality of electrically operated water valves 208, a plurality of water fittings 210, and a plurality of electrical connectors 212. In the preferred embodiment, the water supply 202 is a low pressure water line from a municipal water supply and is in fluid communication with the valve panel 200 using water tubing 203. In at least one embodiment, the water supply comprises a water storage tank with a pump. A water manifold 206 directs water to each of the plurality of electrically operated water valves 208. The water fittings 210 are mounted to the cover of the valve panel 200 and are in fluid communication with the electrically operated water valves 208. The electrical connectors 212 are mounted to the cover of the valve panel 200 and are utilized to electrically connect the controller 104, to the watering zone sensor devices 300. In at least one embodiment the electrical connectors 212 are electrically connected to a signal conditioner in the valve panel 200.

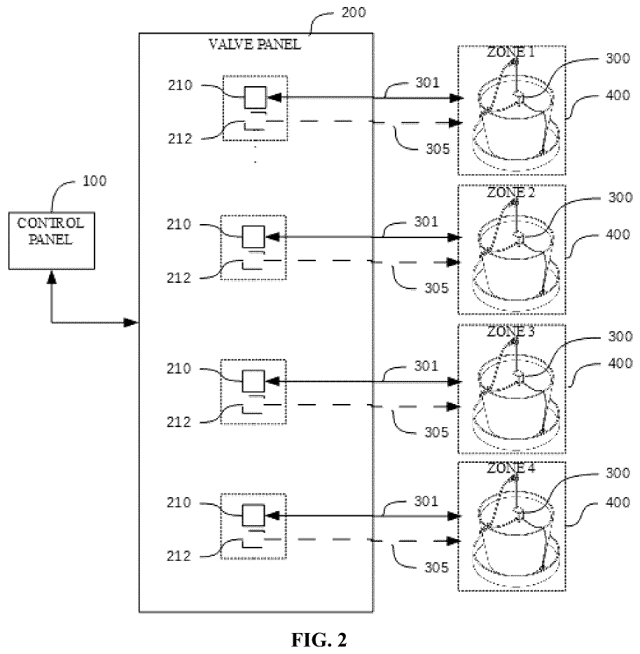

A representative automated plant watering system according to the preferred embodiment showing the control panel 100, valve panel 200, water fittings 210, electrical connectors 212, and a plurality of watering zone sensor devices 300 located in a plurality of watering zones 400 is shown in FIG. 2. The watering zone sensor devices 300 in the watering zones 400 are in fluid communication with the water fittings 210 of the valve panel. The watering zone sensor devices 300 in the watering zones 400 are electrically connected to the electrical connectors 212 of the valve panel.

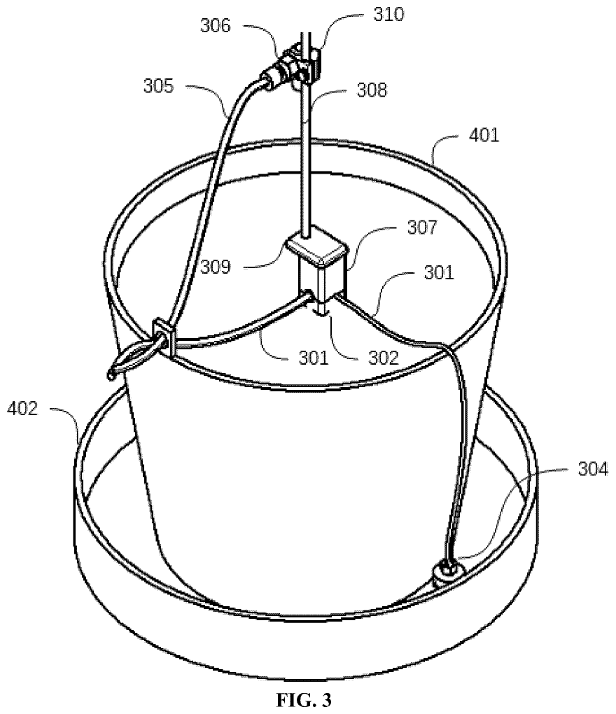

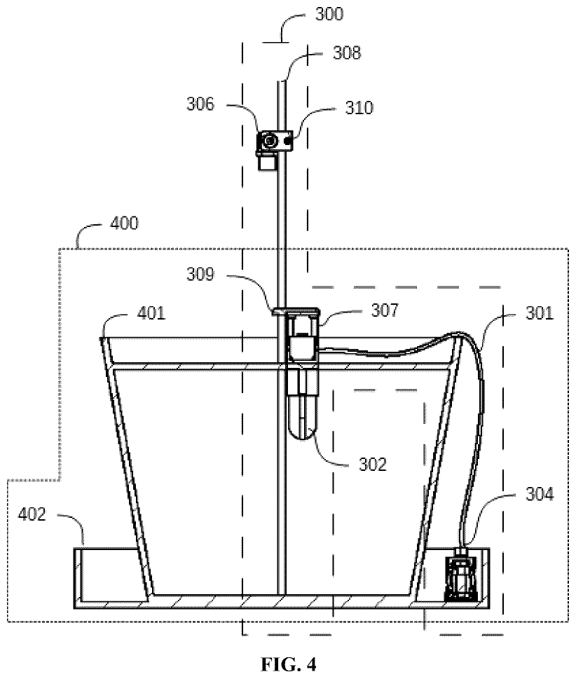

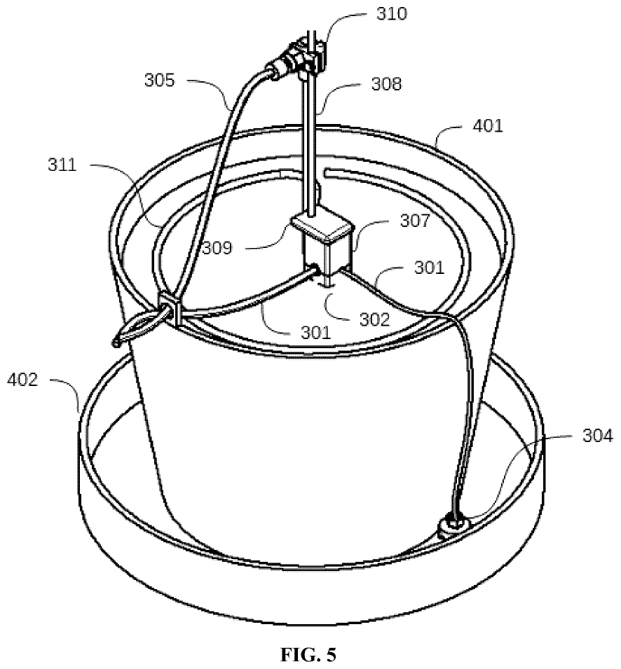

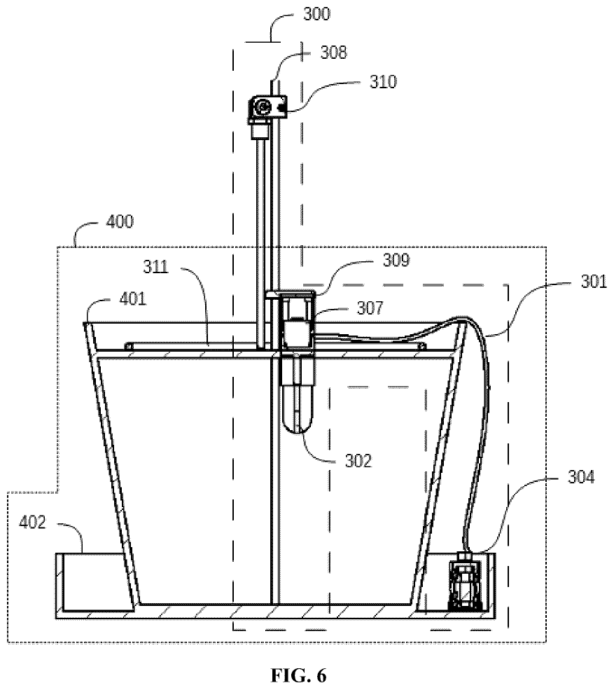

The diagrams in FIG. 3 and FIG. 4 show the preferred embodiment of the watering zone sensor device 300 installed in a watering zone 400. The watering zone 400 comprising a standard plant pot containing soil 401, and a plant saucer 402. The standard plant pot 401 can be any readily available commercial plant pot of any size, shape, or material of construction. The plant pot 401 is positioned in relation to the plant saucer 402 so that the plant saucer collects excess water that drains from the plant pot 401. The watering zone sensor device 300 is comprised of a soil moisture sensor 302, a water level sensor 304, a watering nozzle 306, a waterproof housing 307, a dowel 308, a first retainer 309, and a second retainer 310. The soil moisture sensor 302 and the water level sensor 304 of the watering zone sensor device 300 are electrically connected to the electrical connector 212 of the valve panel 200 using the third multi-conductor electrical cable 301. The watering nozzle 306 of the watering zone sensor device 300 is in fluid communication with the water fitting 210 of the valve panel 200 using water tubing 305. The soil moisture sensor 302 is a capacitance based moisture sensor in the preferred embodiment. In general, capacitance based moisture sensors measure the change in the dielectric constant of the soil between two electrodes, wherein the dielectric constant of the soil changes according to the amount of moisture in the soil. In at least one embodiment, a different type of sensor that detects moisture in soil is utilized in the watering zone sensor device 300. The soil moisture sensor 302 is placed in direct contact with the soil to monitor the moisture level of the soil. The soil moisture sensor 302 is at least partially housed in a waterproof housing 307 and located in the top portion of the soil in the plant pot 401 of the watering zone 400 in the preferred embodiment, but is buried deeper in the soil in other embodiments. In the preferred embodiment, the water level sensor 304 is a float switch that is located within the plant saucer 402, but is located elsewhere in other embodiments, such as mounted to the sidewall of the plant saucer 402. In at least one embodiment, the watering zone sensor device 300 utilizes an alternative water level sensor 304, such as a pressure transducer, to detect a high water level in the plant saucer 402. The waterproof housing 307 is secured to the dowel 308 with the first retainer 309. The watering nozzle 306 is secured to the dowel 308 with the second retainer 310. As shown in FIG. 5 and FIG. 6, in at least one embodiment, the watering zone sensor device 300 utilizes drip irrigation tubing 311 instead of a watering nozzle 306 to provide water to a watering zone 400. The dowel 308 assists in securing the waterproof housing 307 and soil moisture sensor 302 in the soil and secures the watering nozzle 306 in close proximity to the soil moisture sensor 302. The watering nozzle 306 can be adjusted up and down on the dowel 308 using the second retainer 310 in order to adjust the watering nozzle spray diameter to fit different size plant pots 401. The dowel 308 is a round wood dowel in the preferred embodiment, but is a different shape or material in other embodiments.

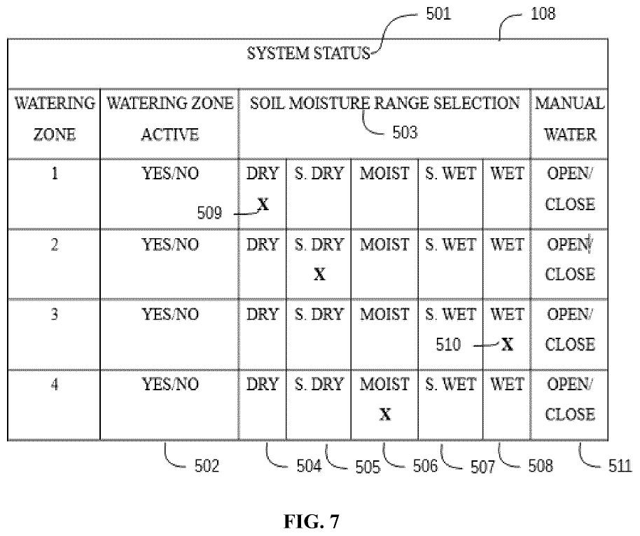

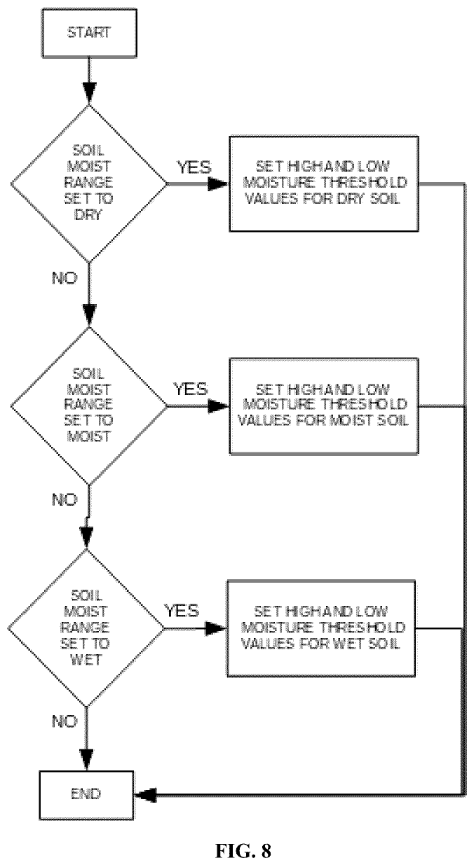

Referring to FIG. 7, a diagram showing the elements of the user interface 108 according to the preferred embodiment. The user interface on the control panel allows the user to activate or inactivate 502 the monitoring and watering of one or more watering zones, select one of a plurality of moisture ranges 503 (dry 504, slightly dry 505, moist 506, slightly wet 507, and wet 508 in the preferred embodiment) for each watering zone 400, open and close watering valves 511 to manually water a watering zone 400, and displays text indicating the status 501 of the system. In at least one embodiment, the user interface 108 includes other elements that allow the user to control and monitor the automated plant watering system, such as including functionality to calibrate the soil moisture sensors. The soil moisture range selection 503 sets the low soil moisture threshold at which to open the valve to begin watering a watering zone and the high soil moisture threshold at which to close the water valve and end watering. A flow chart representing the soil moisture selection process is shown in FIG. 8. When a user selects the dry range 504 for the first watering zone (the selection is labeled 509 in FIG. 7), the controller sets the high and low soil moisture thresholds for the dry range 504 and will activate the watering valve for that location and begin watering when the soil has nearly no moisture and will keep the valve open until the soil is at an adequate level for dry climate plants. In another example, if the user selects the wet range 508 for the third watering zone (the selection is labeled 510 in FIG. 7), the controller sets the high and low soil moisture thresholds for the wet range 508 and will activate the watering valve for that location and begin watering when the soil is slightly moist and will keep the valve open until the soil is completely saturated with water (for plants that require wet soil). In at least one embodiment, the soil moisture range selection 503 employs more or less divisions for various soil moisture ranges. In at least one embodiment, the soil moisture range selection of the graphical user interface utilizes a drop-down menu to list various moisture ranges to choose from. In at least one embodiment, the soil moisture range selection 503 displays a list of plant names to choose from, where the soil moisture thresholds set the low and high values that are most desirable for that particular type of plant.

The watering process in general can be described as follows: during operation, the controller 104 receives signals from the watering zone sensor device 300 that indicate the water level of the plant saucer 401 and the moisture level of the soil in the plant pot 401 of the watering zone 400. If the water level sensor 304 indicates a high water level an alarm 110 is activated. If the water level sensor 104 does not indicate a high water level, the controller 104 then compares the soil moisture sensor 302 signal to the user-selected soil moisture range 503 to open and close the electrically operated water valve 208 to the watering zone 400. If a high water level in the watering zone 400 is detected while watering, the electrically operated water valve 208 is closed, and an alarm 110 is activated.

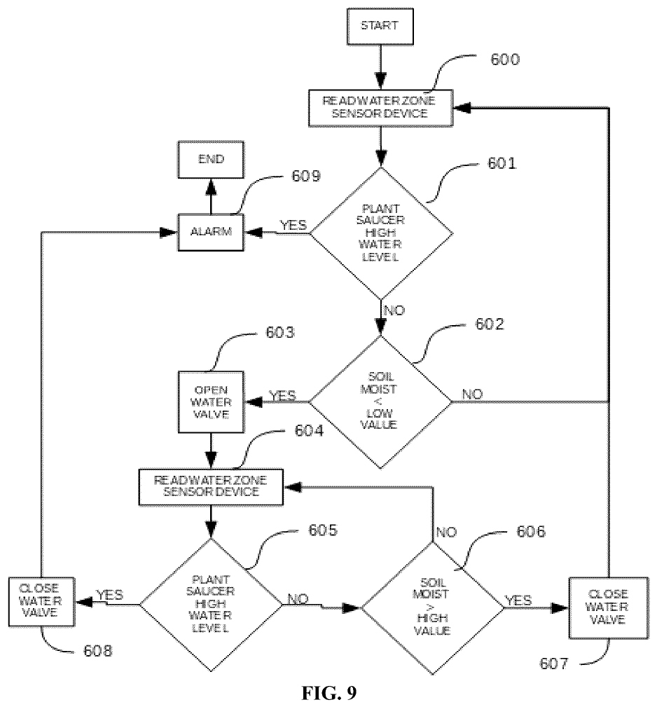

A flow chart showing a representative method for providing water to a watering zone 400 with the automated plant watering system is shown in FIG. 9. In step 600, the controller 104 is programmed to first receive signals from the watering zone sensor device 300. The signals indicates if the plant saucer shows a high water level, and the current soil moisture level of the watering zone 400. In step 601, if the water level sensor 304 indicates a high level of water, the alarm 110 is activated in step 609. If the water level sensor does 304 not indicate a high level of water in step 601, the controller 104 compares the signal from the soil moisture sensor 102 to the low soil moisture threshold in step 602. If the signal indicates that the moisture level of the watering zone 400 is greater than the low threshold, the controller 104 will continue to receive signals pertaining to the condition of the watering zone 400 (step 600). If the signal indicates that the moisture level of the soil in the plant pot 401 of the watering zone 400 is less than the low threshold, the electrically operated water valve 208 is opened in step 603 to begin watering the watering zone 400. The electrically operated water valve 208 now being opened, the controller 104 receives signals from the watering zone sensor device 300 in step 604, if the water level sensor 304 indicates a high water level in the plant saucer 401 in step 605, the electrically operated water valve 208 is closed (step 608), and the alarm 110 is activated (step 609). If the water level sensor 304 does not indicate a high water level in the plant saucer 402, the controller 104 will continue monitoring the soil moisture until the soil moisture level of the soil in the plant pot 401 of the watering zone 400 is greater than the high threshold in step 606, at which point it will close the electrically operated water valve 208 (step 607). After the watering cycle is completed and the electrically operated water valve 208 is closed, the controller 104 will continue monitoring the condition of the watering zone 400 (step 600).

While certain embodiments of the automated plant watering system have been described herein, it is intended that all matter in the detailed description and drawings be interpreted as illustrative rather than in a limiting sense, and that the following claims are intended to cover the various embodiments of the automated plant watering system.

1. An automated plant watering system, comprising:
a control panel;
a valve panel electrically connected to the control panel and in fluid communication with a water source; and
a plurality of watering zone sensor devices electrically connected to the control panel through the valve panel and in fluid communication with the valve panel, each watering zone sensor device of the plurality of watering zone sensor devices comprising a soil moisture sensor and a water level sensor configured to be positioned within a respective watering zone of a plurality of watering zones.

2. The automated plant watering system of claim 1, wherein the respective watering zone comprises:
a plant pot containing soil; and
a plant saucer positioned in relation to the plant pot such that the plant saucer collects excess water that drains from the plant pot.

3. The automated plant watering system of claim 1, wherein the control panel comprises:
a power supply;
a programmable controller electrically connected to the power supply;
an output driver electrically connected to the power supply and electrically connected to the programmable controller;
a user interface electrically connected to the programmable controller; and
an alarm electrically connected to the programmable controller.

4. The automated plant watering system of claim 3, wherein the user interface comprises:
a plurality of inputs for operating the system; and
a display.

5. The automated plant watering system of claim 4, wherein the user interface further comprises a soil moisture range selection for each watering zone of the plurality of watering zones.

6. The automated plant watering system of claim 5, wherein the soil moisture range selection sets a low soil moisture threshold and a high soil moisture threshold for each watering zone of the plurality of watering zones.

7. The automated plant watering system of claim 1, wherein the valve panel comprises:
a manual water valve in fluid communication with the water source;
a water manifold in fluid communication with the manual water valve;
a plurality of electrically operated water valves in fluid communication with the water manifold and electrically connected to the output driver in the control panel;
a plurality of water fittings mounted to the valve panel and in fluid communication with the plurality of electrically operated water valves; and
a plurality of electrical connectors mounted to the valve panel and electrically connected to the programmable controller in the control panel.

8. The watering zone sensor device, comprising:
a soil moisture sensor electrically connected to the electrical connector of the valve panel;
a waterproof housing;
a first retainer;
a water level sensor electrically connected to the electrical connector of the valve panel;
a dowel;
a second retainer; and
a watering nozzle that is in fluid communication with the water fitting of the valve panel.

9. The watering zone sensor device of claim 8, wherein the soil moisture sensor is in direct contact with the soil in the plant pot of the watering zone.

10. The watering zone sensor device of claim 8, wherein the soil moisture sensor is at least partially housed in the waterproof housing.

11. The watering zone sensor device of claim 8, wherein the waterproof housing is attached to the dowel with the first retainer in order to secure the waterproof housing in the soil.

12. The watering zone sensor device of claim 8, wherein the watering nozzle is secured to the dowel with the second retainer in order to keep the nozzle in close proximity to the soil moisture sensor.

13. The watering zone sensor device of claim 8, wherein the watering nozzle can be adjusted up or down on the dowel to increase or decrease the spray diameter to suit the size of the plant pot.

14. The watering zone sensor device of claim 8, wherein the water level sensor is located in relation to the plant saucer so that it can detect a high water level of the plant saucer.

15. The automated plant watering system of claim 3, wherein the programmable controller is programmed to receive signals from each watering zone sensor device pertaining to a soil moisture level of the soil in the plant pot of the respective watering zone and a water level of the plant saucer of the respective watering zone.

16. The automated plant watering system of claim 3, wherein the programmable controller is programmed to compare a soil moisture level of the respective watering zone to the low soil moisture threshold and activate an electrically operated water valve of the valve panel to begin watering the respective watering zone when the soil moisture level is less than the low soil moisture threshold.

17. The automated plant watering system of claim 3, wherein the programmable controller is programmed to compare a soil moisture level of the respective watering zone to the high soil moisture threshold, and deactivate an electrically operated water valve of the valve panel to end watering the respective watering zone when the soil moisture level is greater than the high soil moisture threshold.

18. The automated plant watering system of claim 3, wherein the programmable controller is programmed to deactivate an electrically operated water valve of the valve panel to end watering of the respective watering zone when the water level sensor indicates a high water level in the plant saucer of the respective watering zone.

19. The automated plant watering system of claim 3, wherein the programmable controller is programmed to activate the alarm when the water level sensor indicates a high water level in the plant saucer of the respective watering zone.

20. A method of plant watering, utilizing an automated plant watering system, the method comprising:
receiving signals from each watering zone sensor device of the plurality of watering zone sensor devices, the signals comprising a signal from the soil moisture sensor and a signal from the water level sensor;
activating the alarm if the signal from the water level sensor indicates a high water level in the plant saucer of the respective watering zone;
if the high water level is not indicated, comparing the signal from the soil moisture sensor pertaining to the soil moisture level of the soil in the plant pot to the low soil moisture threshold;
activating an electrically operated water valve of the valve panel when the soil moisture level of the soil in the plant pot is less than the low soil moisture threshold to begin watering of the respective watering zone;
activating the alarm if the high water level in the plant saucer is indicated while watering the respective watering zone;
if the high water level is not indicated, comparing the signal from the soil moisture sensor pertaining to the soil moisture level of the soil in the plant pot to the high soil moisture threshold; and
deactivating the electrically operated water valve of the valve panel when the soil moisture level of the soil in the plant pot is greater than the high soil moisture threshold to end watering of the respective watering zone.
